(19) United States
(12) Patent Application Publication
(10) Pub. No.: US 2021/0291082 A1
(43) Pub. Date: Sep. 23, 2021

(54) BIOREACTOR COMPRISING AN INTERNAL RESONANT VIBRATORY MOTOR FOR AGITATION OF BIODEGRADABLE WASTE COMPRISING HORIZONTAL AND DIAGONAL EXTENSION SPRINGS

(71) Applicant: Xianggen Wu, Mississauga (CA)

(72) Inventor: Xianggen Wu, Mississauga (CA)

(21) Appl. No.: 16/824,861

(22) Filed: Mar. 20, 2020

(51) Int. Cl. B01D 21/28; B01F 11/02; B01D 21/00; B01D 53/02; A01C 23/02; A01C 23/00

(52) U.S. Cl. CPC B01D 21/283; B01F 11/0258; B01D 21/0012; B01D 2253/102; A01C 23/02; A01C 23/008; B01D 53/02

(57) ABSTRACT

The present invention is a resonant vibratory agitation mechanism for installing inside bioreactor containers for agitating and degrading biodegradable waste. It either comprises of a sole layer of horizontally arranged springs with at least one vibration motor installed inside each of the springs, or comprises of a central frame, a plurality of vibration motors fixed on the central frame and a plurality of layers of horizontally or diagonally arranged extension springs. It provides sound waves, vibrations, resonant vibratory frequencies and heat for agitating and degrading biodegradable waste inside a bioreactor container. It saves costs to fabricate a bioreactor container by assembling a plurality of cylindrical drum barrels on top of a receiving tank. A closed-loop recirculation of water, heat, nutrients, O₂ and CO₂ may be established by integrating the present bioreactor system with wicking beds, hydroponics/aeroponics growing beds, a stove unit and a greenhouse.

### CROSS-REFERENCE TO RELATED APPLICATION

This application is a continuation-In-Part of PCT International Application PCT/CA2019/050297 filed on Mar. 11, 2019, which claims priority of the PCT International Application PCT/CA2018/050295 filed on Mar. 12, 2018.

### FIELD OF THE INVENTION

The present invention relates to agitating contents inside containers. More specifically, the present invention relates to agitation of biodegradable waste inside composting bioreactor apparatus that degrades biodegradable waste into liquid and fine particles transportable by circulating water.

### BACKGROUND OF THE INVENTION

Agitation is an important procedure in degrading biodegradable waste inside composting bioreactor containers. Its main purpose is to prevent compaction or lumping of the fed waste and to well aerate all the waste inside a container. In prior art, as shown in patents U.S. Pat. Nos. 5,300,438, 5,744,351 and 9,617,191 either a horizontally rotating mechanism or a vertically rotating mechanism is employed for agitating and mixing purposes.

The above mentioned agitation mechanisms are not good in efficiency and not suitable for some situations for the following reasons: (1) only the torque produced by the driven motor is used for rotating the contents inside a container; (2) the others such us sound waves, vibrations and heat produced by the motor are not useful but burdens that need to be specially managed; (3) the contents inside a container are usually over-agitated, the contents are moved more than required for well de-lumping and aerating; (4) they normally employ high voltage (AC110V or AC220V) powered motors, when solar panels are employed for power source of the motors electricity undergoes loss during inverting from DC12V into AC110V or AC220V; (5) for bioreactor containers such as that of the U.S. Pat. No. 9,617,191, there is not enough space on the top lid for installing an agitation motor for vessels with a width or sectional diameter less than 2.5 feet since there is a feed module sitting on the top lid; (6) these agitators only fit for vertical and horizontal cylinder containers, they don't fit for square or rectangular cuboid containers; and (7) normally only one motor is installed for driving the agitating mechanism, when the only motor is broken it requires an immediate service.

Efforts have been made in employing vibratory resonance for agitating or mixing liquid in sealed containers in pharmaceutical and biological industries. The U.S. Pat. No. 7,195,354 to Vijay Singh disclosed a method of resonant wave mixing for closed containers by a mechanism producing tilting motion to rock a container on a connected platform for mixing ingredients with liquid inside the container. The U.S. Pat. No. 7,188,993 to Harold W Howe etc. disclosed a resonant-vibratory mixing apparatus comprising of a plurality of compression springs and vibration motors connected and supported by frame and mass assemblies.

However, the above resonant mixing mechanisms and methods are for mixing liquid purpose only, they don't fit for installing inside composting bioreactor containers. The operation of rocking or shaking a container is temporary. They are positioned under a container therefore the container normally can not have inlet or outlet ports in working during the resonant mixing operation. They also have the problem of losing energy in the forms of sound waves and heat produced by the driven motors.

It is desirable to have an agitating mechanism that omits the requirement for a space area on the top lid of a bioreactor container, that takes the sound waves, vibrations and heat produced by the driving motor into good uses, that may be driven by DC12V electric power from solar panels, that works well in a standing mode when the inlet and outlet ports of the containers are in operation, and that has one or more backup motors to increase its lifetime without requirement for immediate service.

Comparing with others, the composting bioreactor apparatus disclosed in the patent U.S. Pat. No. 9,617,191 and its continuation-in-part application with application number U.S. Ser. No. 15/615,820 and publication number US-2017-0354906 has the following advantages: (1) it is the first apparatus that integrates both photosynthesis and burning with a stove unit into the composting process, and therefore has extended the definition of conventional composting concept; (2) it is the first apparatus that recycles all biodegradable wastes including solid waste, wastewater and exhaust gases into nutrients to grow food plants; (3) it is the first composting bioreactor that integrates composting process with the Aquaponics technology and therefore leads to the new concept of CompoPonics; and (4) it focuses on degrading the wastes into gases, liquid and fine particles transportable by circulating water, and therefore realises almost completely recycling in high efficiency.

However, besides the aforementioned disadvantages regarding its agitation module, the U.S. Pat. No. 9,617,191 and its related continuation-in-part application U.S. Ser. No. 15/615,820 also have other aspects that need to be improved. (1) The structures of a concaved or conic lower separator and a middle chamber make it complicated in fabricating the bioreactor body vessel, therefore working process of its middle chamber may be diverted partly into its upper chamber and partly into its lower chamber. (2) It consumes a lot of electricity in having a heating-sub-chamber and having all the circulating water flowing through the heating-sub-chamber, normally only the black water containing fecal matter from toilets is required to be sterilized. (3) Eventually, unbreakable humus in its upper chamber may need to be cleaned up every a few years, vertically separated two or more sub-chambers in its upper chamber will make it easier for cleanup operation; when one of the upper chambers is prepared for cleanup the other(s) is/are available for receiving daily waste. (4) When soil inside its wicking bed gets lumped it blocks gases filtering through the soil and is not good for plants to grow; a mechanism is also required to agitate the soil of its wicking bed to keep good state of aeration for roots of plants growing in the wicking bed.

The present invention will provide a new and improved mechanism and method for agitating waste inside composting bioreactor containers and overcome all the aforementioned prior art limitations. It also provides improvements for the U.S. Pat. No. 9,617,191 and its continuation-in-part application U.S. Ser. No. 15/615,820.

### SUMMARY OF THE INVENTION

The present invention is a resonant vibratory agitation mechanism for installing inside composting bioreactor containers or other applications for agitating biodegradable waste, soil or other masses. It either comprises of a sole layer of horizontally arranged springs with at least one waterproof vibration motor installed inside each of the springs, or comprises of a central frame, at least one waterproof vibration motor fixed on a central frame and a plurality of layers of horizontally or diagonally arranged extension springs of which each spring has an outer end connecting with a connecter fixed on side walls inside a container and an inner end connecting with a connecting ring of the central frame, wherein the lowest layer has more springs than each of its upper layers and fed waste are filtered by gaps between any two neighboring springs of a layer, and wherein a vibrational frequency of the springs and the vibrational frequency of the waterproof vibration motor are matched to provide a vibratory resonance.

The present invention fits for containers of both cylindrical shape and square or rectangular cuboid shape. Both low voltage (DC5V or 12V) and high voltage (AC110V or 220V) can be employed for driving the vibration motors. Since the vibration motors stay inside the waste in containers, all the potential energies produced by the vibration motors including sound waves, vibrations and heat are used to agitate and to degrade the waste. Since all the extension springs are connected with the vibration motor via a central frame, energy produced by the vibrations of a vibration motor is amplified by the resident energy of the springs. Coincidences of vibrations of the vibration motors and the springs create resonant vibratory frequencies that have energy to help agitating and degrading the waste.

The present invention also provides the following other improvements for bioreactor apparatus of the U.S. Pat. No. 9,617,191 and its continuation-in-part application U.S. Ser. No. 15/615,820: (1) having the resonant vibratory agitator, the size of the bioreactor vessel can be a container with a width or sectional diameter less than 2.5 feet, since it is not required to have an area on the top lid for installing an agitation motor; (2) comparing with making a whole body vessel vertically with three chambers, fabricating a bioreactor container by sitting a plurality of drums/barrels on a receiving tank not only saves manufacture costs but also makes it easier to transport and to clean up; and (3) it saves costs of electricity to have only the black water rather than all the circulating water sterilized by heating it to 70-100° C.

Other objects, features, and advantages of the present invention will be readily appreciated from the following description. The description makes references to the accompanying drawings, which are provided for illustration of the preferred embodiments. However, such embodiments do not represent the full scope of the invention.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1 shows a vertically sectional elevation of a multi-layer resonant vibratory agitator 30 and a sole-layer resonant vibratory agitator 70 installed inside a bioreactor container which has an upper chamber and a lower chamber;

FIG. 2A shows a horizontally sectional elevation of springs of a horizontal layer of a resonant vibratory agitator inside a vertical cylindrical bioreactor container;

FIG. 2B shows a horizontally sectional elevation of springs of a horizontal layer of a resonant vibratory agitator inside a vertical cuboid bioreactor container;

FIG. 2C shows a vibration motor waterproof treated by sealing a vibrator, a hollow cup motor and part of its wires inside a metal tube for installing inside springs;

FIG. 3A shows perspective of a central frame with two horizontal flat plate surfaces for fixing vibration motors for a vertical cylindrical bioreactor container;

FIG. 3B shows perspective of a central frame with two horizontal flat plate surfaces for fixing vibration motors for a vertical cuboid bioreactor container;

FIG. 3C shows perspective of a central frame with two vertical portrait flat plate surfaces for fixing vibration motors for a vertical cylindrical bioreactor container;

FIG. 3D shows perspective of a central frame with two vertical landscape flat plate surfaces for fixing vibration motors for a vertical cylindrical bioreactor container;

FIG. 3E shows a top view of a multi-layer resonant vibratory agitator 30 which has a top diagonally arranged layer and a second diagonally arranged layer and each layer is composed of 18 extension springs inside a vertical cylindrical container;

FIG. 3F shows a bottom view of a multi-layer resonant vibratory agitator 30 which has a bottom horizontally arranged layer composed of 36 extension springs inside a vertical cylindrical container;

FIG. 4A shows perspective of a bioreactor container comprising of two vertical drums as two upper chambers sitting on top and inside of a rectangular cuboid receiving tank as a lower chamber;

FIG. 4B shows a top view of a receiving tank that has 4 circular openings on its top wall and a plurality of supports inside its inside volume for holding 4 drums as 4 upper chambers;

FIG. 4C shows a vertically sectional elevation of a bioreactor container that has two vertical drums serving as two vertically separated upper chambers of which each upper chamber has a multi-layer resonant vibratory agitator 30 installed;

FIG. 4D shows a vertically sectional elevation of a bioreactor container that has two vertical drums serving as two vertically separated upper chambers of which each upper chamber has a multi-layer resonant vibratory agitator 30 installed, and one of the drums is configured for receiving black water containing fecal matter from toilets;

FIG. 4E shows a vertically sectional elevation of a bioreactor container that has two vertical drums serving as two vertically separated upper chambers of a larger height, of which each upper chamber is installed with a sole-layer resonant vibratory agitator 70 and a multi-layer resonant vibratory agitator 30 with 3 connection rings and 4 vibration motors on its central frame;

FIG. 5A shows a vertically cross-sectional elevation of a wicking bed having a multi-layer resonant vibratory agitator 30 installed inside its top growing media;

FIG. 5B shows a vertically sectional elevation of a wicking bed with large length having 3 multi-layer resonant vibratory agitators 30 installed inside its top growing media;

FIG. 6 shows perspective of an embodiment of the present invention, an integrated bioreactor system for installing in urban household backyards for recycling kitchen waste into organic produce;

FIG. 7 shows a vertically sectional elevation of another embodiment of the present invention, an integrating bioreactor system additionally having a stove, an air carbon filter, at least one inflatable gas storage vessel, a water heating tank and a rainwater collector kit;

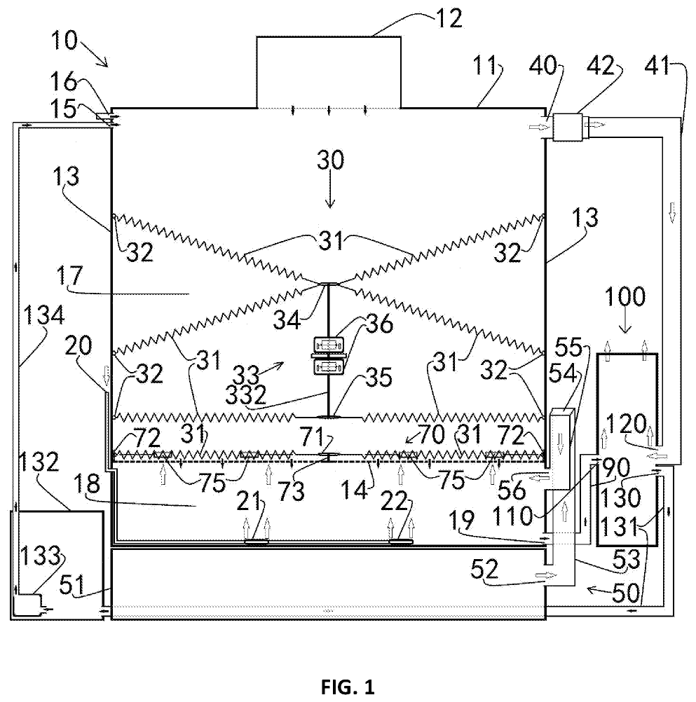

FIGS. 1, 7, 4C-E and 5A-B also shows flow charts for both gases and liquid re-circulating amongst an integrated wicking bed, a stove unit and a bioreactor container etc., wherein bold arrows show flowing direction of liquid while hollow arrows show flowing direction of gases.

### DETAILED DESCRIPTION OF THE DRAWINGS

As shown in FIG. 1, a multi-layer resonant vibratory agitator 30 is installed inside a bioreactor container 10 which has a perforated plate separator 14 separating its inside volume into an upper chamber 17 and a lower chamber 18. The multi-layer resonant vibratory agitator 30 stays in the upper chamber 17. The perforated plate separator 14 has a plurality of filter holes or gaps for filtering liquid and particles.

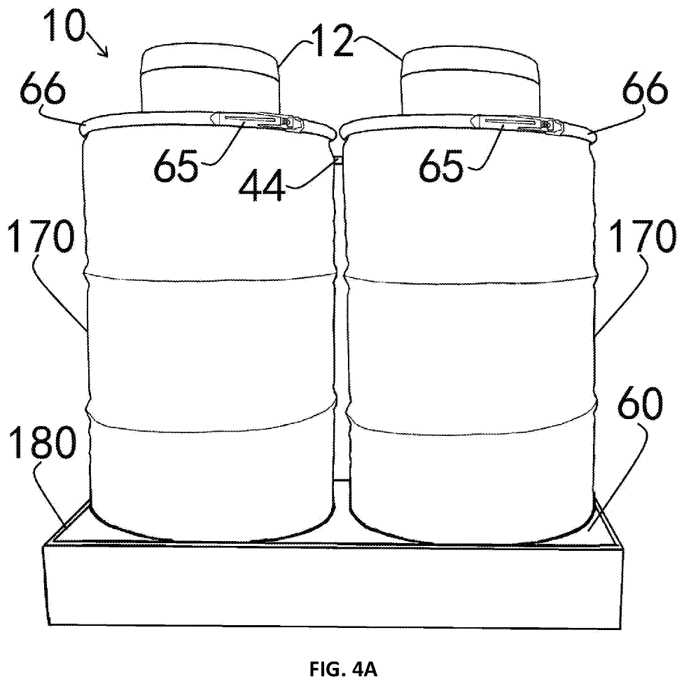

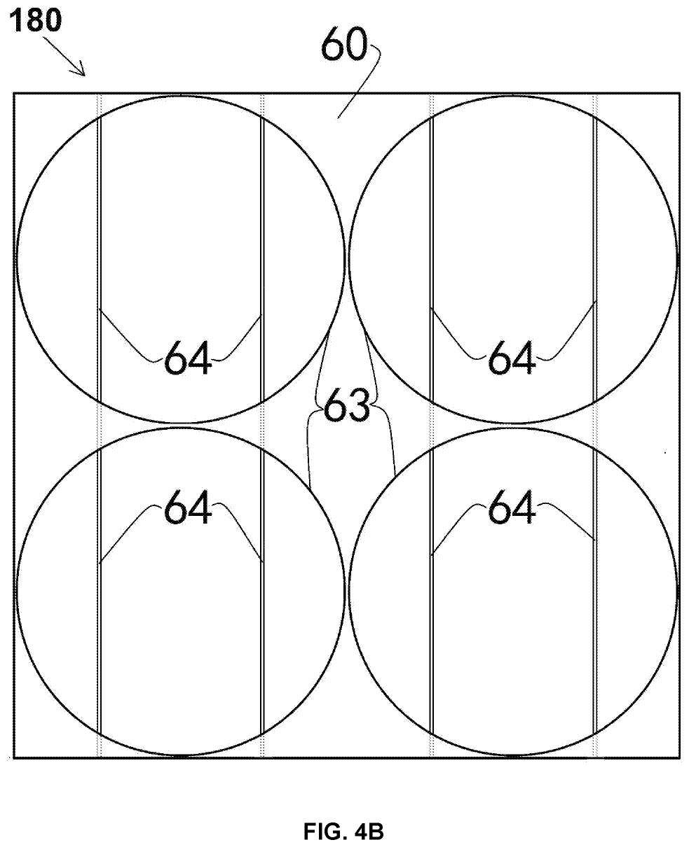

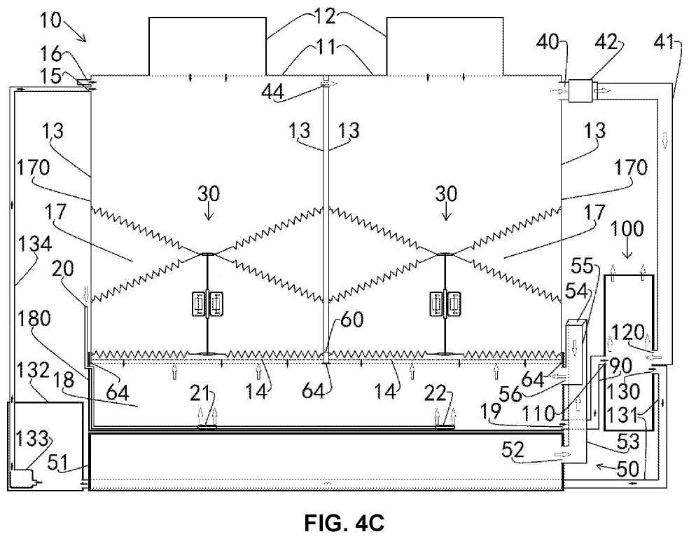

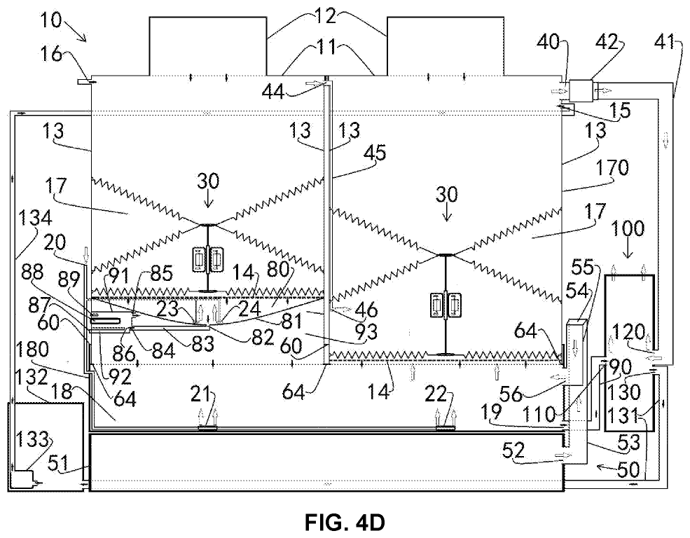

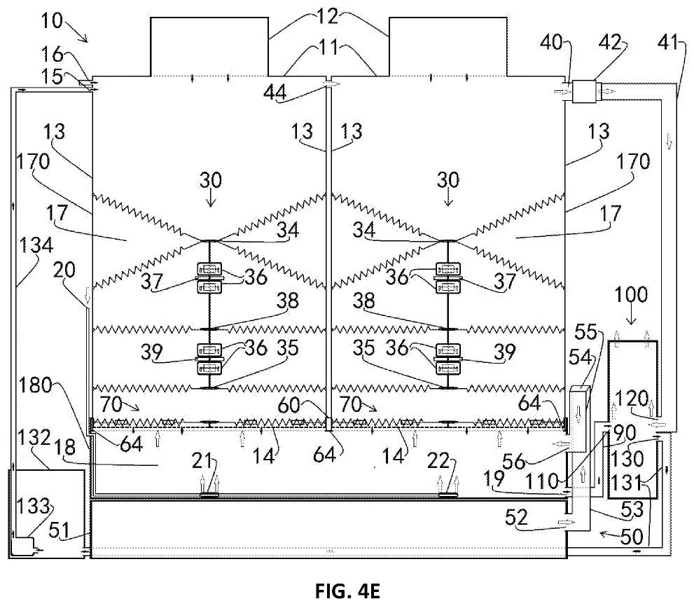

As shown in FIGS. 1, 4C-E and 5A-B, a multi-layer resonant vibratory agitator 30 comprises at least one vibration motor 36 and a plurality of layers of horizontally or diagonally arranged extension springs 31 of which each of the springs 31 has an outer end connecting with a connecter 32 fixed on side walls 13 inside the upper chamber 17 and an inner end connecting with one of the connecting rings 34-35 of a central frame 33 on which the vibration motors 36 are fixed, wherein a vibrational frequency of the springs 31 and the vibrational frequency of the waterproof vibration motor 36 are matched to provide vibratory resonance. Preferably, in a multi-layer resonant vibratory agitator 30, a lower layer may have more springs 31 than its upper layer so that the fed waste is filtered by gaps between any two neighboring springs of a layer. With this filtering function, larger sized waste stays in the upper layer while smaller sized waste filters into the lower layer inside the upper chamber 17.

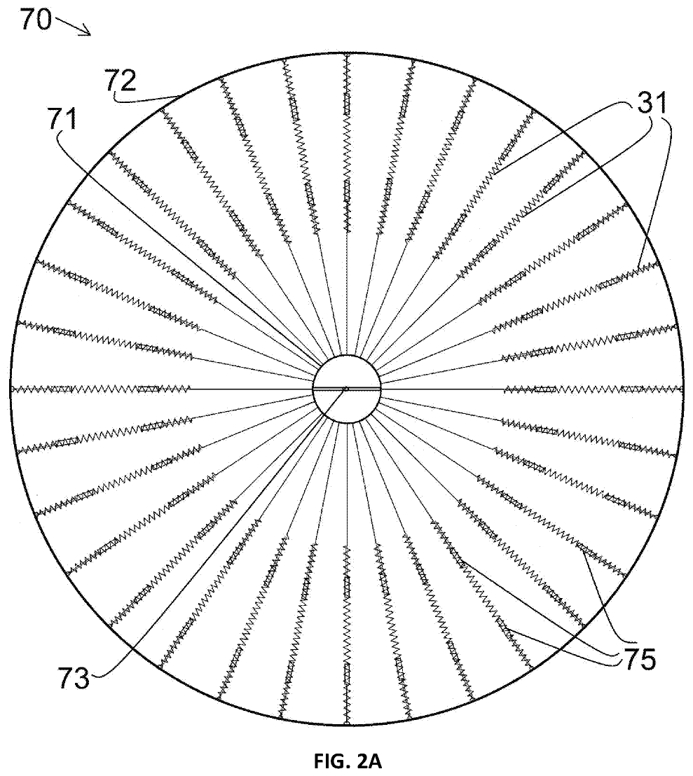

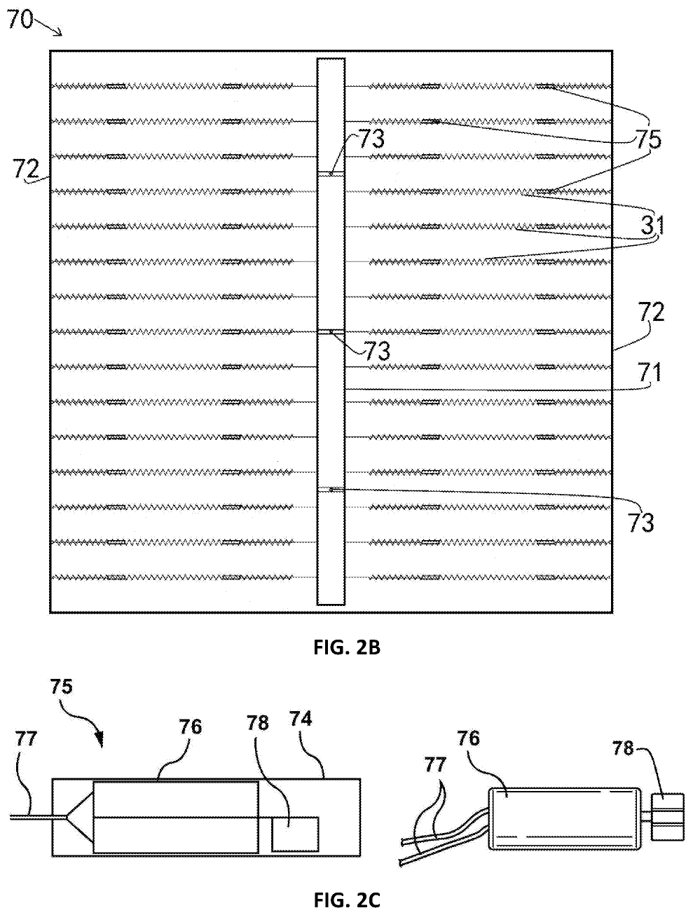

As shown in FIG. 2A-B, in a multi-layer resonant vibratory agitator 30 fitting either for a vertical cylindrical shaped container 10 or for a vertical cuboid shaped container 10, all springs 31 of a typical horizontal layer have the same length and the same quantity of coils.

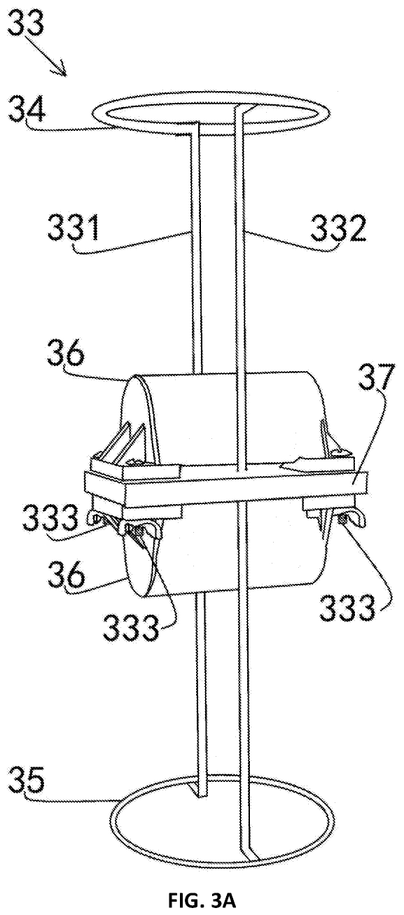

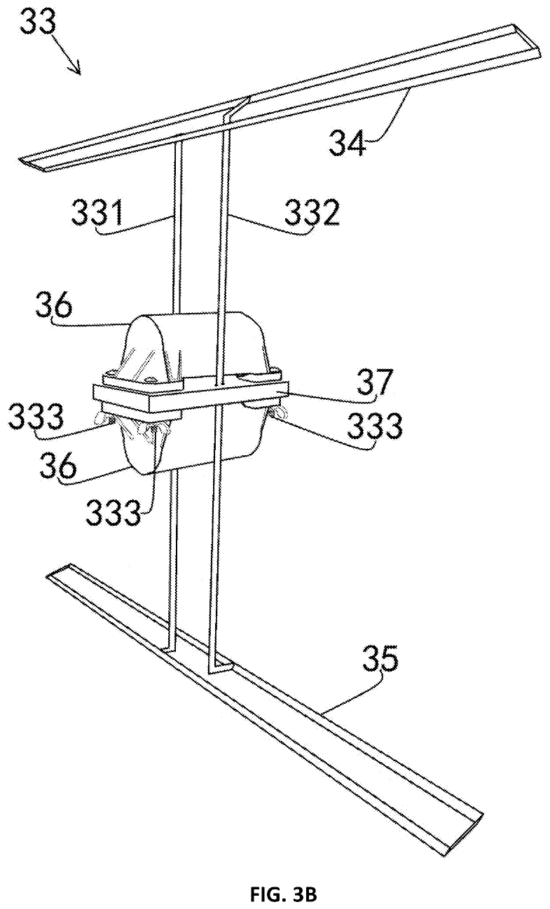

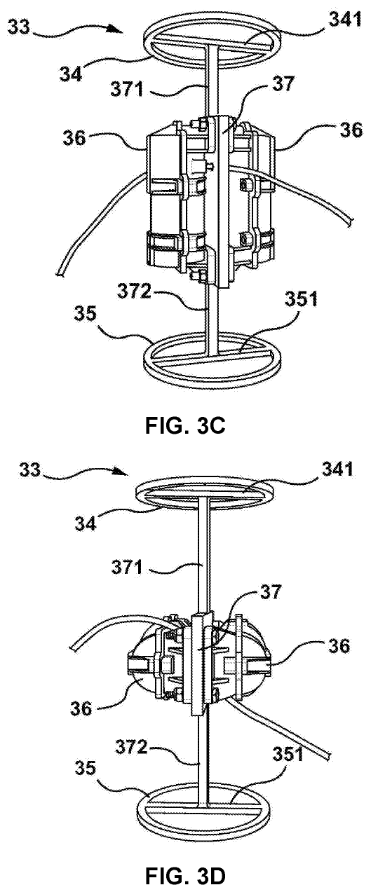

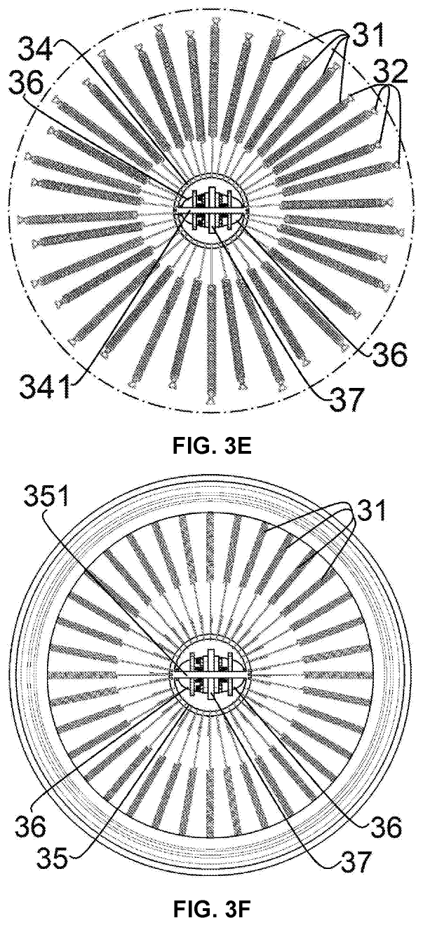

As shown in FIGS. 3A-B, a central frame 33 is a substantial metal frame. It has either circular rings 34-35 for vertical cylindrical container 10 or rectangular rings 34-35 for vertical cuboid container 10 at its top end and its bottom end for connecting the inner end of each of the extension springs 31. It also has at least one horizontal flat plate 37 between the rings 34-35 for fixing vibration motors 36 on each of its two flat surfaces by use of screw bolts 333. The rings 34-35 and the flat plate 37 are substantially welded with vertical connecting rods 331-332.

Preferably, as shown in FIGS. 3C-D, the flat plate 37 of the central frame 33 for a vertical cylindrical shaped container 10 may be arranged either as a vertical portrait position as shown in FIG. 3C or as a vertical landscape position as shown in FIG. 3D. In this case, the flat plate 37 is substantially welded with a central rod 341 of top ring 34 by way of an upper connecting rod 371, and with a central rod 351 of bottom ring 35 by way of a lower connecting rod 372.

As shown in FIGS. 1, 3E and 4C-E, a multi-layer resonant vibratory agitator 30 inside an upper chamber 17, has diagonally arranged uppermost two layers of springs 31 symmetrically balanced so that the top ring 34 of the central frame 33 stays in a vertical position parallel to the opposite a vertical middle point between the uppermost two layers of connecters 32 of springs 31 while a conical top shape is formed along the upper surface of the springs 31 of the uppermost layer.

As shown in FIGS. 1, 3F and 4C-E, inside a bioreactor container 10 a horizontal layer of extension springs 31 is positioned above and near to an upper surface of a perforated plate separator 14 so that vibrations of the springs 31 may help prevent the filter holes of the perforated plate separator 14 from blocking by silt or sticky particles. This layer of extension springs 31 is either the lowest layer of a multi-layer resonant vibratory agitator 30 as shown in FIGS. 3F and 4C-D, or an independent sole-layer resonant vibratory agitator 70 as shown in FIGS. 1 and 4E. The vertical gap between the lower edge of each of the spring 31 and the upper surface of the perforated plate separator 14 is less than one inch.

As shown in FIGS. 1, 2A-C and 4E, a sole-layer resonant vibratory agitator 70 has one horizontal layer of springs 31 and stays above and near to the upper surface of the perforated plate separator 14. It is pre-assembled so that it is easy to be installed on the upper surface of the perforated plate separator 14. It has an outer frame 72 to stay along the inside surface of side walls 13 and an inner frame 71 to be fixed on the perforated plate separator 14 with a bolt/bolts 73. Each spring 31 has an inner end connected with the inner frame 71 and an outer end connected with a connector or a hole on the outer frame 72. The height of the outer frame 72, the relative vertical position of each connector or hole on the outer frame 72 and the height of fix bolt(s) 73 for fixing the inner frame 71 are coordinated to keep the vertical gap between the lower edge of each spring 31 and the upper surface of the perforated plate separator 14 less than one inch. Inside each spring 31 at least one vibration motor 75 is installed to provide vibrations and resonant vibratory frequencies for agitating the waste above the perforated plate separator 14 and for speeding up degrading the waste of the space volume into fine particles to filter into the lower chamber 18. As shown in FIG. 2C, the vibration motor 75 is of waterproof by having a hollow cup motor 76 with a vibrator 78 and part of its wires 77 sealed inside a metal tube 74. The hollow cup motor 76 is of low voltage (≤DC12V) and small sized (with sectional diameter≤10 mm and length 25 mm) so that springs with inner diameter less than 12 mm can be employed for the sole-layer resonant vibratory agitator 70. Preferably, two or more vibration motors 75 are employed inside each of the springs 31 so that the vibration motors 75 in each of the springs 31 are configured either to work together to increase vibration strength or to have half as working motor(s) and the other half as backup motor(s) to increase lifetime of the sole-layer resonant vibratory agitator 70.

As shown in FIG. 4E, to fit bioreactor containers with upper chambers of a very large vertical height, the central frame 33 of a multi-layer resonant vibratory agitator 30 may have at least one more additional connecting ring 38 between the top ring 34 and bottom ring 35 for adding more layers of springs 31, and at least one more flat plate 39 among the rings 34, 35 and 38 for fixing more vibration motors 36. The two vibration motors 36 on each of the two surfaces of the flat plates 37 and 39 may be configured either to work together to increase vibration strength or to have one set as a working motor while the other set as a back-up motor to increase lifetime of the multi-layer resonant vibratory agitator 30.

The vibration motor 36 is of waterproof by sealing its motor, vibrator(s) and part of its wires inside a plastic or metal shell. The vibration motors 36 installed on the central frame 33 are configured with relatively higher torque and lower rotation speed (for example less than 6,000 RPM), so that each of the connected springs 31 is driven to vibrate in a relatively lower frequency with longer vibration wave length for reaching more space volume around the springs 31. The vibration motors 75 installed inside the springs 31 of the sole-layer resonant vibratory agitator 70 are configured with lower torque and higher rotation speed (for example with a zero-load rotation speed more than 40,000 RPM), so that each of the springs 31 of the sole-layer resonant vibratory agitator 70 is driven to vibrate in higher frequency with shorter vibration wave length to reach relatively less space volume around the springs 31 of the lowest layer, to speed up degrading the waste near to the upper surface of the perforated plate separator 14 into fine particles to filter into the lower chamber 18. The vibration motors 36 and vibration motors 75 may be configured either for both kinds to work together or for each kind to work in different time zones.

As shown in FIGS. 1, 2C and 4E, the horizontally positioned vibration motors 75 are at the same height level as the liquid level for introducing into an integrated wicking bed 100 from a bioreactor container 10. The vibration motors 75 are submerged in the liquid so that heat from high speed rotations of the hollow cup motors 76 is quickly released through its metal tube 74 into the liquid around the vibration motors 75.

As shown in FIGS. 1, 2A-B and 3A-B, a bioreactor container 10 to be installed with a multi-layer resonant vibratory agitator 30 or a multi-layer resonant vibratory agitator 30 plus a sole-layer resonant vibratory agitator 70 may be of vertical cylindrical shape or of vertical cuboid shape. It may be fabricated by positioning a substantial perforated plate separator 14 inside to form an upper chamber 17 and a lower chamber 18. The size of the container 10 may be big or small depending on the quantity of waste to be treated. However, containers 10 with a width or diameter of bigger than 3 feet are too heavy for one person to transport and too big to access most backyard gates.

Preferably, as shown in FIGS. 4A-E and 6, a bioreactor container 10 may be fabricated by sitting at least one drum 170 with a sectional diameter of around 2 feet on top or inside of a receiving tank 180. The drum(s) 170 and the receiving tank 180 can be transported separately and are easy to access all backyard gates. The inside volume of the tank 180 serves as a lower chamber 18. Each inside volume of drums 170 serves as an upper chamber 17. Each bottom wall of drums 170 either having pre-drilled holes or gaps serves as a perforated plate separator 14 or being cut out has an additional perforated plate separator 14 fixed on it. The receiving tank 180 has a plurality of circular openings 63 on its top wall 60 and a plurality of supports 64 inside its volume 18 for holding drums 170. Therefore, the bioreactor container 10 may have a plurality of relatively separated upper chambers 17. All the upper chambers 17 may be configured either for each to receive different kind of waste or for each to receive all kinds of waste at different time zone. Every a few years the unbreakable humus inside an upper chamber 17 may need to be cleaned up, when one drum 170 is preparing for cleanup, the other drum(s) 170 can still receive daily waste. The drum 170 may be removed from the top wall 60 during cleaning up operation and be position back after cleaning up operation. Preferably, a vent pipe 44 between any two neighboring drums 170 is employed to lead exhaust gases from all other drums 170 to exit from an exhaust gas outlet port 40 on one of the drums 170. The liquid inlet ports 15-16 may be either both on one drum 170 or each on one of the drums 170. The contact areas between bottom end side walls of drums 170 and top edges of circular openings 63 are well sealed to prevent leaks of liquid, odor and exhaust gases.

As shown in FIGS. 1 and 4A-E, the bioreactor container 10 has at least one top wall 11 having a feed module 12 attached on. Preferably, the top wall 11 may be openable for the purpose to reach inside the upper chamber 17 to clean up unbreakable humus. As shown in FIG. 4A, for a cylindrical drum 170, its top edge and an outer edge of the top wall 11 may be tightened or opened up by using a closing ring 66 with a fastening mechanism 65.

As shown in FIGS. 1, 4C-E, 5A-B, at least one wicking bed 100 is integrated with a bioreactor container 10 for further degrading the liquid discharged from the container 10 and for supplying water, nutrients and heat to the plants growing inside the wicking bed 100. The bioreactor container 10 has at least two liquid inlet ports 15-16, the inlet port 15 is for receiving recirculating water from an integrated wicking bed 100 while the inlet port 16 is for receiving wastewater from other resources such as from kitchen sinks. The liquid mix filtered into the lower chamber 18 includes water introduced into the upper chamber 17 by way of the liquid inlet ports 15-16, water produced from degradation of the waste in the upper chamber 17, and fine particles filtered through the perforated plate separator 14 from the upper chamber 17. As shown by the bold arrows, liquid exiting a liquid outlet port 19 is introduced by way of a pipe 90 into an inlet port 110 of the integrated wicking bed 100 to supply water, heat and nutrients to food plants 150 growing in the wicking beds 100. Liquid exiting from a liquid outlet port 130 of the wicking bed 100 is introduced into a sump tank 132 by way of water pipes 131. A water pump 133 is installed inside the sump tank 132 to introduce water by pipe 134 into the bioreactor container 10 through the liquid inlet port 15. Therefore, a closed-loop water recirculation is established between the bioreactor container 10 and the integrated wicking bed 100.

As shown in FIGS. 1, 4C-E and 5A-B, an aeration module 20 has aerators 21-22 installed inside the lower chamber 18 to supply oxygen to both the lower chamber 18 and the upper chambers 17 to support aerobic organisms for degrading the waste. Preferably, an integrated system has a stove unit 50 that has a heat radiator 51 staying under the bioreactor container 10 as its support base and supplying heat to the container 10. As shown by the hollow arrows, flue gas from the stove unit 50 is introduced into the lower chamber 18 by way of an exhaust gas inlet port 56. Flue gas of the stove unit 50 flows through the heat radiator 51, an outlet port 52 of the heat radiator 51, a pipe 53, a U-turn pipe 54, a pipe 55 and the gas inlet port 56 into the lower chamber 18. The U-turn pipe 54 is positioned in a higher level than the liquid level inside the bioreactor container 10 to prevent the liquid refluxing into the pipe 53. The flue gas from the stove unit 50 undergoes “washed” by the liquid in the lower chamber 18, filtered by the waste in the upper chamber 17, exiting the upper chamber 17 together with exhaust gases produced from degradation of the waste inside the bioreactor container 10 through the exhaust gas outlet port 40, flowing through an inline duct fan 42 and duct 41, entering into an integrated wicking bed 100 through its exhaust gas inlet port 120, further washed by liquid in an upper channel 101, further filtered by a top growing media 190 in the wicking bed 100, and lastly exiting from the top growing media 190 into atmosphere. If the wicking bed 100 is staying inside a greenhouse (not shown), CO₂ of the exhaust gases exiting from the top growing media 190 serves as a nutrient for the plants 150 growing in the wicking bed 100. Oxygen produced by the plants may also serves as a component for combustion inside a combustion chamber (not shown) of the stove unit 50. When a vent pipe (not shown) is configured to introduce air into the combustion chamber from the greenhouse in which the integrated wicking bed 100 stays, a closed-loop gas recirculation may be established among the stove unit 50, the bioreactor container 10 and plants growing in the wicking bed 100 inside the greenhouse. The duct fan 42 positioned between the exhaust gas outlet port 40 of the bioreactor container 10 and the exhaust gas inlet port 120 of the wicking bed 100 plays an important role for recirculating the flue gas. It pushes air inside duct 41 into the upper channel 101 of the wicking bed 100 to cause a positive pressure inside the upper channel 101 therefore pushing the exhaust gases inside the upper channel 101 to filter through the top growing media 190. It also draws air from the bioreactor container 10 to cause a negative pressure inside the container 10 therefore drawing flue gas flowing from the heat radiator 51 through the lower chamber 18, the upper chamber 17, the exhaust gas outlet port 40, and the duct fan 42 itself into the duct 41.

As shown in FIG. 4D, one of the upper chambers 17 may be configured for receiving black water containing fecal matter from toilets. The perforated plate separator 14 is positioned upward and a middle chamber 80 and lower volume 93 having a heating sub-chamber 91 are added by fixing a concaved or conic separator 81 immediately under the perforated plate separator 14 on an inner surface of the side walls 13. The heating sub-chamber is positioned between the concaved or conic separator 81 and the top wall 60 of the receiving tank 180. Wastewater filtered through the perforated plate separator 14 is collected in the middle chamber 80; it then flows through an outlet port 82 at the central lowest area of the middle chamber 80 and a pipe 83 into the heating sub-chamber 91 by way of an inlet port 84; and lastly, it exits an outlet port 85 of the heating sub-chamber 91 and enters into the lower chamber 18. An electric heater 88 and a bimetal temperature control switch 89 are installed inside the heating sub-chamber 91 from outside of the side wall 13. Vertically, the outlet port 85 of the heating sub-chamber 91 is in a higher position than its inlet port 84, therefore, all wastewater flowing through the heating sub-chamber is heated by the electric heater 88. Working temperature inside the heating sub-chamber 91 is set at 70-100° C. for killing pathogenic organisms and is controlled by the bimetal temperature control switch 89. The heated wastewater from the heating sub-chamber 91 is to be moderated in temperature by the liquid inside the lower chamber 18, therefore, liquid introduced into the wicking bed 100 is in a right temperature fitting for the growing plants 150. The heating sub-chamber 91 also has a second outlet port 86 to connect by way of a pipe 92 into an outlet port 87 on the side wall 13 positioned between the heating sub-chamber 91 and the top wall 60 of the receiving tank 180, so that wastewater inside the middle chamber 80, heating sub-chamber 91 and the connecting pipes 83 and 92 may be emptied to prevent them from breaking by icing during winter season.

As shown in FIG. 4D, the aeration module 20 connects into both the aerators 21-22 inside the lower chamber 18 and the aerators 23-24 inside the middle chamber 80 to supply oxygen to the middle chamber 80 and the upper chamber 17 for receiving black water. A vent pipe 44 connecting into the neighboring upper chamber 17 is further connecting into a pipe 45, so that exhaust gases from the upper chamber 17 for receiving black water are introduced into the lower layer of the neighboring upper chamber 17 by way of an exit 46 of the pipe 45, to be further filtered by the waste inside the neighboring upper chamber 17 before the gases exit the neighboring upper chamber 17 through the exhaust gas outlet 40.

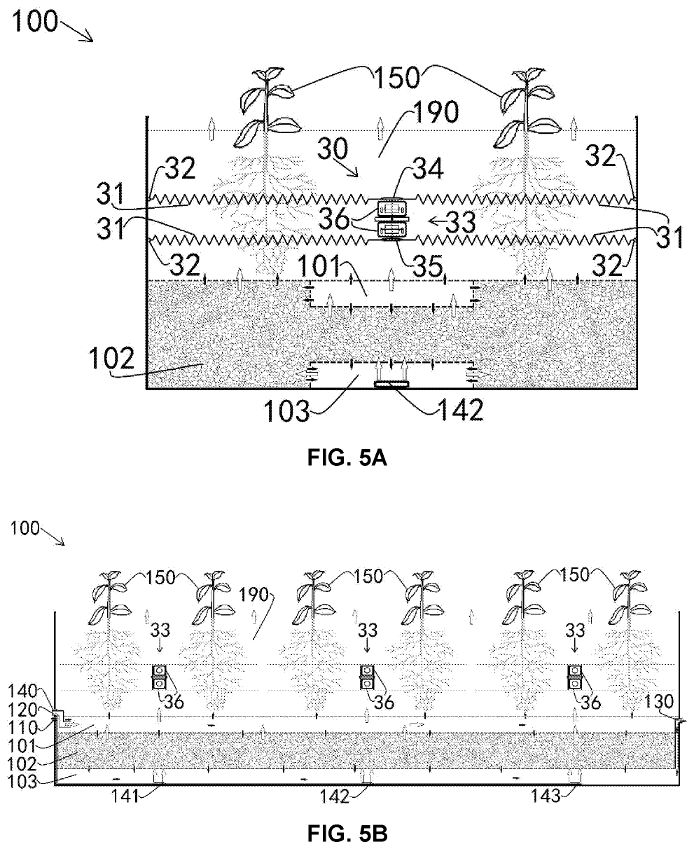

As shown in FIG. 5A, a multi-layer resonant vibratory agitator 30 may be installed inside a wicking bed 100 to provide vibrations to loosen its top growing media 190 and to improve aeration around roots of plants 150. As shown in FIG. 5B, a plurality of resonant vibratory agitators 30 may be installed inside a wicking bed 100 with very large length.

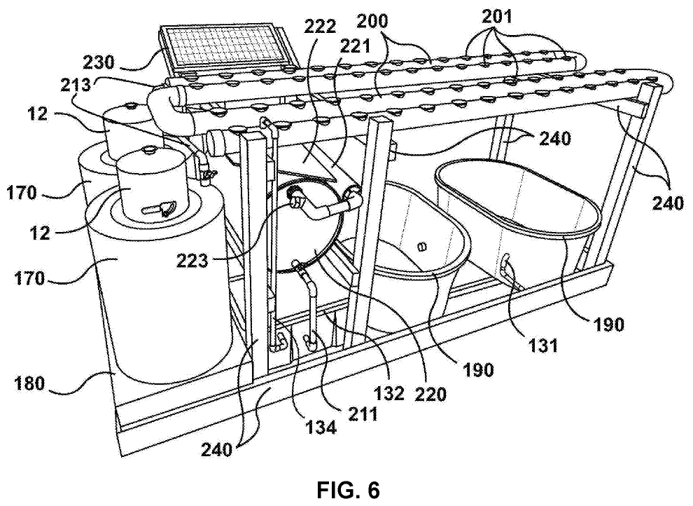

As shown in FIGS. 1, 4C-E and 5A-B, a wicking bed 100 has an upper layer of 8-12 inches filled with top growing media 190 for growing plants 150, and a lower layer of 8-12 inches having an upper channel 101, an lower channel 103 and a middle channel 102 filled with bio-filter media. Both the gas duct 41 connecting with the exhaust gas outlet port 40 of a bioreactor container 10 and the liquid pipe 90 connecting with the liquid outlet port 19 of the bioreactor container 10 are introduced into the upper channel 101 through its gas inlet port 120 and its liquid inlet port 110. A second aeration module 140 connects into aerators 141-143 in the lower channel 103. The wicking bed supplies by wicking into the growing plants 150 with water, nutrients, heat and oxygen from its upper channel 101. The liquid introduced into the upper channel 101 is further filtered and degraded by the bio-filter media inside the middle channel 102. The liquid filtered into the lower channel 103 exits the wicking bed 100 through a liquid outlet port 130 which is directly connected into the lower channel 103 at the other end of the wicking bed 100. The liquid from the liquid outlet port 130 is introduced either into another wicking bed 100 or some hydroponic growing pipes 200 as shown in FIG. 6, or into a sump tank 132 and further into the liquid inlet port 15 of the bioreactor container 10 to establish a closed-loop liquid recirculation.

As shown in FIG. 6, an embodiment of the present invention for installing in urban household backyards for recycling kitchen waste into organic produce has the following components: (1) two drums 170 each having a feed module 12 sitting on its top wall, a perforated plate separator 14 fixed on its bottom wall (not shown), a multi-layer resonant vibratory agitator 30 (not shown) installed inside each inside volume 17 (not shown); (2) a receiving tank 180 to receive filtered liquid and particles from the drums 170; (3) at least one tank 190 to be employed and configured as a wicking bed 100; (4) at least one layer of hydroponic growing pipes 200 staying above the tanks 190 having a plurality of openings to hold net cups 201 for growing plants; (5) a sump tank 132 having a water pump 133 (not shown) to introduce liquid from the sump tank 132 into the hydroponic growing pipes 200 by way of a pipe 134, and having an automatic water level control valve (not shown) installed on its side wall to automatically add water into the sump tank 132 when water level inside the sump tank 132 is lower than the level of automatic water level control valve; (6) a water reservoir tank 220 staying above the sump tank 132, connected into the automatic water level control valve inside the sump tank 132 by way of a pipe 211, and having a roof board 222 to collect rain water by way of pipe 221 which has an overflow port 223 to discharge extra water; (7) at least one air-pump (not shown) to supply air into inside the receiving tank 180, the drums 170 and the tanks 190; (8) a solar panel 230 having a solar charge controller (not shown) and a battery (not shown) to supply electricity to the water pump, air-pump and the motors of the multi-layer resonant vibratory agitators 30 inside the drums 170; and (9) a plurality of wood frames 240 to support the whole integrated system and to keep the components in the right positions for establishing a closed-loop water recirculation. Liquid fed into the drums 170 and produced from degradation of the fed waste inside the drums 170 flows through the receiving tank 180, the tanks 190, the sump tank 132, the hydroponic growing pipes 200 and lastly back into the drums 170 by way of a pipe 213. When the water level inside the sump tank 132 is lower than the automatic water level control valve, it automatically adds water from the water reservoir tank 220 which collects rainwater from the roof board 222. The sump tank 132 also has an overflow port (not shown) vertically positioned between its top lid and the horizontal level of the automatic water level control valve to discharge extra water of the sump tank 132.

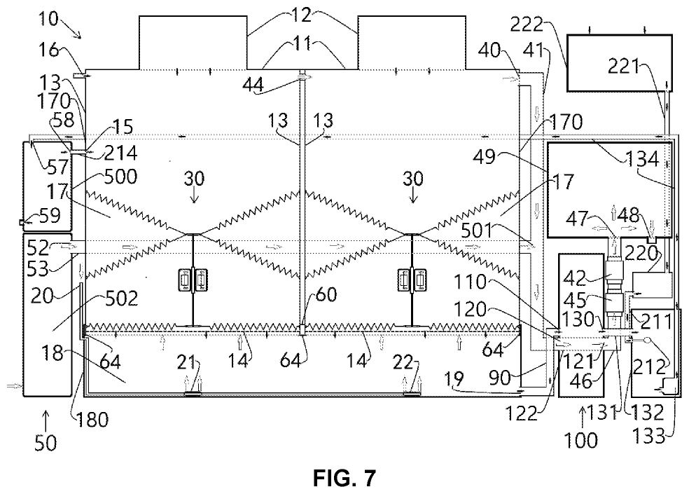

As shown in FIG. 7, another embodiment of the present invention is an integrated bioreactor system having the following components: (1) two drums 170 each having a feed module 12 sitting on its top wall, a perforated plate separator 14 fixed on its bottom wall, a multi-layer resonant vibratory agitator 30 installed inside each volume 17; (2) a receiving tank 180 to receive filtered liquid and particles from the drums 170; (3) at least one wicking bed 100; (4) a sump tank 132 having a water pump 133 to introduce liquid from the sump tank 132 into a heating tank 500 by way of a pipe 134, and having an automatic water level control valve 212 installed on its side wall to automatically add water into the sump tank 132 when water level inside the sump tank 132 is lower than the level of automatic water level control valve 212; (5) a water reservoir tank 220 staying above the sump tank 132, connected into the automatic water level control valve inside the sump tank 132 by way of a pipe 211, and having a roof board 222 to collect rain water by way of pipe 221; (6) a stove unit having a combustion chamber 502 for receiving and combusting a biomass waste, and having a chimney duct 53 connecting into its outlet port 52 for introducing its flue gas into the upper channel 101 through the gas inlet port 120 at a first end of the wicking bed 100 after being integrated into a T-connector 501 of the gas duct 41; (7) a duct pipe 122 inside the upper channel 101 for introducing the flue gas from the gas inlet port 120 into a gas outlet port 121 at the second end of the wicking bed 100 and for releasing heat of the flue gas into liquid inside the upper channel 101; (8) an inline duct fan 42 connecting into the gas outlet port 121 of the wicking bed 100 via an air carbon filter 45 and a duct 46, and into at least one inflatable gas storage vessel 49 via a second gas inlet port 47 of the inflatable gas storage vessel 49; (9) said inflatable gas storage vessel 49 having at least one gas outlet port 48 having a connected valve manifold with valves and pressure monitors (not shown) for dispersing its inside stored filtered flue gas into an onsite closed planting space and for pumping its inside stored filtered flue gas into a portable gas storage tank (not shown); and (10) at least one air-pump (not shown) to supply air into inside volumes of the receiving tank 180, the drums 170, the wicking bed 100 and the sump tank 132. Liquid fed into the drums 170 and produced from degradation of the fed waste inside the drums 170 flows through the receiving tank 180, the wicking bed 100, the sump tank 132, the heating tank 500 and lastly back into the drums 170 by way of a pipe 214. When the water level inside the sump tank 132 is lower than the automatic water level control valve 212, it automatically adds water from the water reservoir tank 220 which collects rainwater from the roof board 222. The sump tank 132 also has an overflow port (not shown) vertically positioned between its top lid and the horizontal level of the automatic water level control valve to discharge extra water of the sump tank 132. The biomass waste fed into the combustion chamber is combusted and degraded into light, heat, ash and flue gas. The heat is mainly used to heat the liquid inside the heating tank 500 on top of the stove unit to a temperature of 70-100° C. for killing pathogen microorganisms. The sterilized liquid inside the heating tank 500 may be either introduced into an onsite integrated hydroponics/aeroponics plant device (not shown) or discharged into a portable liquid storage tank (not shown) via a second liquid outlet port 59 of the heating tank 500. The ash may be applied inside the wicking bed 100 or other integrated wetland growing beds (not shown). The flue gas is to be stored inside the inflatable gas storage vessel 49 after being cooled by liquid inside the upper channel 101 and filtered by the air carbon filter 45. The stored filtered flue gas may be used to supply CO₂ to grow plants either by dispersing directly into an onsite closed planting space or by pumping into a portable gas storage tank for using in offsite closed planting spaces.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function and manner of operation, assembly and use are deemed readily apparent and obvious to those skilled in the art, and all equivalent relationships to those illustrated in the drawings and described in the specification are intended to be encompassed by the present invention.

What is claimed is:

1. A multi-layer resonant vibratory agitator inside an upper chamber of a bioreactor container having a perforated plate separator to separate its inside volume into said upper chamber to receive biodegradable waste and a lower chamber to receive liquid and particles generated in said upper chamber, comprising:
a. a plurality of layers of horizontally arranged connectors fixed on an inner surface of side walls inside said upper chamber of said bioreactor container;
b. a central frame having a top ring, a bottom ring, at least one connecting rod between and substantially welded with said top ring and said bottom ring, and at least one area on said connecting rod for mounting a waterproof vibration motor;
c. a plurality of layers of horizontally or diagonally arranged extension springs wherein each of said springs having an inner end connecting with either said top ring or said bottom ring of said central frame and an outer end connecting with one of said connectors on said side walls; and
d. at least one said waterproof vibration motor mounted on said area of said connecting rod of said central frame;
whereby said multi-layer resonant vibratory agitator provides at least one of sound waves, vibrations, resonant vibratory frequencies and heat to agitate said biodegradable waste inside said upper chamber and to speed up degrading said biodegradable waste into liquid and particles transportable by a circulating water.

2. The multi-layer resonant vibratory agitator of claim 1, wherein said central frame further having at least one flat plate substantially welded on said connecting rod and each flat plate having two opposite flat surfaces for mounting one said waterproof vibration motor on each of said two flat surfaces, wherein said flat plate is either horizontally positioned, vertically portrait positioned or vertically landscape positioned, and whereby said two waterproof vibration motors on each said flat plate either to work together to increase vibration strength, or to have one set as a working motor and the other set as a backup motor to increase lifetime of said multi-layer resonant vibratory agitator.

3. The multi-layer resonant vibratory agitator of claim 1, wherein said central frame further having at least one additional connecting ring substantially welded on said connecting rod between said top ring and said bottom ring to provide connections for additional horizontal or diagonal layers of springs.

4. The multi-layer resonant vibratory agitator of claim 1, wherein said springs having a first vibrational frequency matching with a second vibrational frequency of said waterproof vibration motor, whereby vibrations generated by said waterproof vibration motor are amplified by a resident energy of said springs and a vibratory resonance is generated for agitating said biodegradable waste inside said upper chamber to speed up degrading said biodegradable waste into liquid and particles transportable by a circulating water.

5. The multi-layer resonant vibratory agitator of claim 1, wherein said layers of horizontally or diagonally arranged extension springs whereof a lower layer has more springs than its upper layer, whereby said biodegradable waste fed into said upper chamber is filtered by gaps between any two neighboring springs of a layer and therefore larger sized waste stays in upper layer while smaller sized waste filters into lower layer inside said upper chamber.

6. The multi-layer resonant vibratory agitator of claim 1, wherein said layers of diagonally arranged extension springs further having two layers of springs whereof each spring has an inner end connecting with said top ring of said central frame and an outer end connecting with either one of said connectors of an uppermost layer or one of said connectors of a lower layer on said side walls, wherein said two layers of springs are symmetrically balanced, whereby said top ring of the central frame stays in a vertical position parallel to a vertical middle point between said connectors of said uppermost layer and said connectors of said lower layer for keeping the central frame in a stable and balanced position, and whereby a conical top shape is created along an upper surface of said uppermost layer of springs for receiving said biodegradable waste fed into said upper chamber.

7. The multi-layer resonant vibratory agitator of claim 1, wherein said layers of horizontally arranged springs further having a lowest layer staying above said perforated plate separator of said bioreactor container with a vertical gap of less than 2.5 cm between a lower edge of said lowest layer of springs and an upper surface of said perforated plate separator, whereby vibrations of said lowest layer of springs prevent filter holes of said perforated plate separator from blocking by silt or sticky particles.

8. A sole-layer resonant vibratory agitator fixed on an upper surface of a perforated plate separator inside a bioreactor container having said perforated plate separator separating its inside volume into an upper chamber for receiving biodegradable waste and a lower chamber for receiving liquid and particles generated in said upper chamber, comprising:
a. an outer frame along an inner surface of side walls of said upper chamber;
b. a plurality of connectors or holes on said outer frame;
c. an inner frame to be fixed on said upper surface of said perforated plate separator;
d. one layer of horizontally arranged springs having an inner end connecting with said inner frame and an outer end connecting with one of said connectors or holes on said outer frame; and
e. at least one waterproof vibration motor installed inside each of said springs;
whereby said sole-layer resonant vibratory agitator provides at least one of vibrations, sound waves, resonant vibratory frequencies and heat to agitate a biodegradable waste in a volume above and near to said perforated plate separator and to speed up degrading said biodegradable waste into liquid and fine particles transportable by a circulating water.

9. The sole-layer resonant vibratory agitator of claim 8, further having two or more waterproof vibration motors installed inside each of said springs, whereby all waterproof vibration motors inside each of said springs are configured either to work together to increase vibration strength or to have half set as working motor(s) and the other half set as backup motor(s) to increase lifetime of said sole-layer resonant vibratory agitator.

10. The sole-layer resonant vibratory agitator of claim 9, wherein each of said waterproof vibration motors inside each of said springs is waterproof treated by sealing a vibrator, a hollow cup motor and part of its wires inside a metal tube, wherein said hollow cup motor is of low voltage (12V) and has a zero-load rotation speed of more than 40,000 RPM, a cross section diameter of less than 10 mm and a length of less than 25 mm.

11. The sole-layer resonant vibratory agitator of claim 10, wherein said waterproof vibration motors further stay in a bioreactor container having a horizontal liquid level to submerge said waterproof vibration motors for preventing said vibration motors from overheating.

12. A bioreactor system for recycling biodegradable waste, comprising:
a. a plurality of cylindrical drums for receiving biodegradable waste;
b. a receiving tank for receiving liquid and particles generated in said drums;
c. said multi-layer resonant vibratory agitator of claim 1 or said multi-layer resonant vibratory agitator of claim 1 plus said sole-layer resonant vibratory agitator of claim 8 inside each of said drums;
d. a feed module on a top wall of each of said drums for feeding said biodegradable waste;
e. a perforated plate separator attached to an opened bottom wall of each of said drums for filtering said liquid and particles generated in each of said drums into said receiving tank;
f. at least one liquid inlet port on a side wall or on said top wall of at least one of said drums;
g. a liquid outlet port on a side wall of said receiving tank;
h. an aeration module having aerators installed inside said receiving tank; and
i. a plurality of circular openings on a top wall of said receiving tank and a plurality of supports inside said receiving tank for holding said drums, wherein gaps between bottom end side walls of said drums and top edges of said circular openings of said top wall are sealed from leaking liquid, odor and gases;
whereby said bioreactor system degrades said biodegradable waste into said liquid and particles for supplying into a planting bed.

13. The bioreactor system of claim 12, further having at least one integrated wicking bed, comprising:
a. a container having an upper layer of 20-30 cm filled with a top growing media and a lower layer of 20-30 cm having an upper channel, a lower channel and a middle channel filled with a bio-filter media;
b. a second aeration module having aerators installed inside said lower channel;
c. a liquid inlet port for introducing said liquid and particles from said liquid outlet port of said receiving tank into said upper channel;
d. a liquid outlet port connecting into said lower channel for introducing a further filtered liquid either into another integrated wicking bed or into a sump tank; and
e. said sump tank having a water pump having a connecting pipe for introducing said further filtered liquid into said liquid inlet port of at least one of said drums;
whereby said bioreactor system having an established closed-loop liquid recirculation supplies said liquid and particles into said integrated wicking bed for growing plants.

14. The bioreactor system of claim 13, further having a solar panel, a battery and a solar charger controller to supply electricity to drive said water pump, said aeration modules, said multilayer resonant vibratory agitators, and said sole-layer resonant vibratory agitators.

15. The bioreactor system of claim 13, further having at least one layer of hydroponic growing pipes with a plurality of openings to hold net cups for growing plants staying above said wicking bed, and having said connecting pipe of said water pump introducing said further filtered liquid into said hydroponic growing pipes, wherein said hydroponic growing pipes having a second connecting pipe to discharge said further filtered liquid into said liquid inlet port of at least one of said drums, whereby said bioreactor system having an established closed-loop liquid recirculation supplies said liquid and particles into said integrated wicking bed and said hydroponic growing pipes for growing plants.

16. The bioreactor system 13, further having a water reservoir tank staying above said sump tank to store rain water from a roof board via a third connecting pipe between said water reservoir tank and said roof board, wherein said water reservoir tank having a discharge pipe connecting into an automatic water level control valve fixed on a side wall of said sump tank, wherein said third connecting pipe having an overflow outlet port to discharge extra water of said water reservoir tank and wherein said sump tank having an overflow port to discharge extra water of said sump tank, and whereby said water reservoir tank automatically collecting rain water and automatically adding water into the sump tank when water level of the sump tank is lower than said automatic water level control valve.

17. The integrated bioreactor system of claim 13, further having:
a. an exhaust gas outlet port on said side wall or said top wall of one of said drums;
b. a vent pipe between any two neighboring side walls of said drums for introducing an exhaust gas from all other drums into the drum having said exhaust gas outlet port;
c. a gas inlet port connecting into said upper channel of said lower layer of said wicking bed, and
d. an inline duct fan positioned between and having a duct connected with said gas inlet port of said wicking bed and said exhaust gas outlet port of one of said drums for introducing said exhaust gas from said drums into said upper channel of said wicking bed;
whereby said bioreactor system introduces said exhaust gas from said drums into said wicking bed for further filtering and for supplying CO₂ into growing plants.

18. The bioreactor system of claim 13, further having at least one multi-layer resonant vibratory agitator of claim 1 installed inside said top growing media of said wicking bed for loosening said top growing media to improve aeration around plant roots.

19. The bioreactor system of claim 13, wherein at least one of said drums is configured for receiving a black water containing fecal matter, comprising:
a. an inside volume of said drum separated into an upper chamber, a middle chamber and a lower volume;
b. said perforated plate separator to separate said upper chamber from said middle chamber, and a concaved or conic separator to separate said middle chamber from said lower volume;
c. said top wall and said feed module on said top wall for receiving said biodegradable waste;
d. at least one liquid inlet port on said side wall of said upper chamber for receiving said black water;
e. said multi-layer vibratory agitator of claim 1 or said multi-layer vibratory agitator of claim 1 plus said sole-layer resonant vibratory agitator of claim 8 installed inside said upper chamber;
f. said pipe vent connecting into a pipe inside a neighboring drum for introducing said exhaust gas from said upper chamber into a lower layer of said neighboring drum;
g. said aeration module having aerators installed inside said middle chamber;
h. a liquid outlet port in a central lowest area of said concaved or conic separator for introducing said black water received or generated in said upper chamber and collected in said middle chamber into a heating sub-chamber; and
i. said heating sub-chamber inside said lower volume having:
i. an electric heater and a bimetal temperature control switch, whereby said electric heater is controlled ON/OFF by said bimetal temperature control switch according to changes of temperature inside said heating sub-chamber,
ii. an inlet port for receiving said black water from said middle chamber,
iii. an outlet port for introducing a heated black water into said receiving tank, and
iv. a second outlet port for introducing said black water into an outlet port below the heating sub-chamber on a side wall of said lower volume, whereby said black water inside the middle chamber, the heating sub-chamber and all connecting pipes in the lower volume may be emptied to prevent said connecting pipes from breaking by icing during winter season;
whereby said black water received and generated in the upper chamber undergoes collected in the middle chamber, introduced into the heating sub-chamber, heated inside the heating sub-chamber to a temperature of 70-100° C. to kill pathogenic organisms, introduced into the receiving tank, moderated in temperature inside the receiving tank, and lastly supplied into said wicking bed for growing plants.

20. The bioreactor system of claim 17, further having a stove unit having a radiator positioned under said receiving tank as its support base and having a second duct for introducing a flue gas of said stove unit from an outlet port of said radiator into said receiving tank by way of an exhaust gas inlet port on a second side wall of said receiving tank, whereby said flue gas supplies heat into said bioreactor system and supplies CO₂ into said plants inside said wicking bed after being “washed” by said liquid inside the receiving tank and by said liquid inside the upper channel of the wicking bed, and being filtered by said biodegradable waste in the drums and by said top growing media in the wicking bed.

21. The bioreactor system of claim 13 or claim 17, further having
a. a stove unit comprising
i. a combustion chamber for receiving and combusting a biomass waste,
ii. a chimney duct connecting into said gas inlet port for introducing a flue gas generated in said combustion chamber into a duct pipe inside said upper channel at a first end of said wicking bed,
iii. a first gas outlet port connecting into said duct pipe inside said upper channel at a second end of said wicking bed,
iv. an air carbon filter for filtering said flue gas having a first end connecting into said first gas outlet port of said wicking bed and a second end connecting into a first end of an inline duct fan,
v. said inline duct fan having a second end connecting into a second gas inlet port of an inflatable gas storage vessel for driving a filtered flue gas into said inflatable gas storage vessel,
vi. said inflatable gas storage vessel for storing said filtered flue gas having a second gas outlet port connecting into a valve manifold, and
vii. said valve manifold having valves and pressure monitors for dispersing a stored filtered flue gas into an onsite closed planting space and for pumping said stored filtered flue gas into a portable gas storage tank; and
b. a heating tank on top of said stove unit for heating said further filtered liquid to kill pathogen microorganisms having a liquid inlet port for receiving said further filtered liquid from said sump tank, a first liquid outlet port for discharging a sterilized liquid into a portable liquid storage tank, and a second liquid outlet port for introducing said sterilized liquid either into said drums or into an integrated hydroponics/aeroponics planting device;
whereby said stove unit converts said biomass waste into heat energy for heating to sterilize said further filtered liquid; whereby said flue gas generated from said combustion chamber supplies heat and CO₂ into onsite growing plants after being cooled by said liquid inside the upper channel of the wicking bed, filtered by the air carbon filter and stored inside said inflatable gas storage vessel; and whereby said stored filtered flue gas is further pumped into portable gas storage tanks for offsite planting uses.
